(19) United States
(12) Patent Application Publication
(10) Pub. No.: US 2013/0025596 A1
(43) Pub. Date: Jan. 31, 2013

(54) METHODS AND SYSTEMS FOR MODEL-BASED TRANSFORMED PROPORTIONAL ASSIST VENTILATION

(71) Applicants: Mehdi M. Jafari, Laguna Hills, CA (US); Peter R. Doyle, Vista, CA (US); Rhomere S. Jimenez, San Diego, CA (US); Jeffrey K. Aviano, Escondido, CA (US); Edward R. McCoy, Vista, CA (US); Gail F. Upham, Fallbrook, CA (US)

(72) Inventors: Mehdi M. Jafari, Laguna Hills, CA (US); Peter R. Doyle, Vista, CA (US); Rhomere S. Jimenez, San Diego, CA (US); Jeffrey K. Aviano, Escondido, CA (US); Edward R. McCoy, Vista, CA (US); Gail F. Upham, Fallbrook, CA (US)

(73) Assignee: Nellcor Puritan Bennett LLC, Boulder, CO (US)

(21) Appl. No.: 13/191,709

(22) Filed: Jul. 27, 2011

(51) Int. Cl. A61M 16/00

(52) U.S. Cl. 128/204.23

(57) ABSTRACT

This disclosure describes systems and methods for providing a model-based transformed proportional assist breath type during ventilation of a patient. The disclosure describes a novel breath type that delivers a target pressure calculated based on a predetermined trajectory and a support setting to a triggering patient.

### INTRODUCTION

Medical ventilator systems have long been used to provide ventilatory and supplemental oxygen support to patients. These ventilators typically comprise a source of pressurized oxygen which is fluidly connected to the patient through a conduit or tubing. As each patient may require a different ventilation strategy, modern ventilators can be customized for the particular needs of an individual patient. For example, several different ventilator modes have been created to provide better ventilation for patients in various different scenarios.

### Model-Based Transformed Proportional Assist Ventilation

This disclosure describes systems and methods for providing a model-based transformed proportional assist breath type during ventilation of a patient. The disclosure describes a novel breath type that delivers a target pressure calculated based on a predetermined trajectory (representing a physiologic-based functional morphology) and a support setting to a triggering patient.

In part, this disclosure describes a method for ventilating a patient with a ventilator. The method includes:

a) monitoring at least one patient parameter;

b) detecting an inspiratory trigger based on the at least one monitored patient parameter;

c) receiving a predetermined pressure trajectory;

d) calculating a target pressure based at least on the predetermined pressure trajectory and a support setting; and

e) delivering the target pressure to a patient based on the detected inspiratory trigger.

Yet another aspect of this disclosure describes a ventilator system that includes: a pressure generating system adapted to generate a flow of breathing gas; a ventilation tubing system including a patient interface for connecting the pressure generating system to a patient; one or more sensors operatively coupled to at least one of the pressure generating system, the patient, and the ventilation tubing system, a trajectory module determines a pressure trajectory; a MT-PA module, and a processor in communication with the pressure generating system, the one or more sensors, the trajectory module, and the MT-PA module. At least one sensor of the one or more sensors is capable of generating an output indicative of an inspiration flow. The MT-PA module calculates at least one target pressure based at least on the pressure trajectory and a support setting. Further, the MT-PA module utilizes the output indicative of the inspiration flow to determine a patient trigger for delivery of a breath to the patient.

The disclosure further describes a computer-readable medium having computer-executable instructions for performing a method for ventilating a patient with a ventilator. The method includes:

a) repeatedly monitoring at least one patient parameter;

b) repeatedly detecting an inspiratory trigger based on the at least one monitored patient parameter;

c) repeatedly receiving a predetermined pressure trajectory;

d) repeatedly calculating a target pressure based at least on the predetermined pressure trajectory and a support setting; and

e) repeatedly delivering the target pressure to a patient based on the detected inspiratory trigger.

The disclosure also describes a ventilator system including means for monitoring at least one patient parameter; means for detecting an inspiratory trigger based on the at least one monitored patient parameter; means for receiving a predetermined pressure trajectory; means for calculating a target pressure based at least on the predetermined pressure trajectory and a support setting; and means for delivering the target pressure to a patient based on the detected inspiratory trigger.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments of systems and methods described below and are not meant to limit the scope of the invention in any manner, which scope shall be based on the claims appended hereto.

FIG. 1 illustrates an embodiment of a ventilator.

FIG. 2 illustrates an embodiment of a method for ventilating a patient on a ventilator.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to control the percentage of oxygen in the gas supplied by the ventilator to the patient. Further, as each patient may require a different ventilation strategy, modern ventilators can be customized for the particular needs of an individual patient. For example, several different ventilator breath types have been created to provide better ventilation for patients in various different scenarios.

Effort-based breath types, such as proportional assist (PA) ventilation, determine a dynamic profile of ventilatory support derived from continuous estimation of patient effort and respiratory characteristics. This desired dynamic profile is computed in real- or quasi-real-time and used by the ventilator as a set of points for control of applicable parameters.

Initiation and execution of an effort-based breath, such as PA, has two operation prerequisites: (1) detection of an inspiratory trigger; and (2) detection and measurement of an appreciable amount of patient respiratory effort to constitute a sufficient reference above a ventilator's control signal error deadband. Advanced, sophisticated triggering technologies detect initiation of inspiratory efforts more efficiently. In ventilation design, patient effort may be represented by the estimated inspiratory muscle pressure and is calculated based on measured patient inspiration flow. Patient effort is utilized to calculate a target pressure for the inspiration.

A PA breath type refers to a type of ventilation in which the ventilator acts as an inspiratory amplifier that provides pressure support based on the patient's effort. The degree of amplification (the “support setting”) is set by an operator, for example as a percentage based on the patient's effort. In one implementation of a PA breath type, the ventilator may continuously monitor the patient's instantaneous inspiratory flow and instantaneous net lung volume, which are indicators of the patient's inspiratory effort. These signals, together with ongoing estimates of the patient's lung compliance and lung/airway resistance and the Equation of Motion, allow the ventilator to estimate a patient effort and derive therefrom a target pressure to provide the support that assists the patient's inspiratory muscles to the degree selected by the operator as the support setting. The support setting input by the operator divides the total work of breathing calculated between the patient and the ventilator as shown in the equations below:

P_{P}(t)=(1.0−k)*(total support); and 1)

P_{V}(t)=k*(total support). 2)

P_{P} is the amount of pressure that must be provided by the patient at a time t, P_{V} is the amount of pressure provided by the ventilator at the time t, total support is the sum of contributions by the patient and ventilator, and k is the support setting (percentage of total support to be contributed by the ventilator) input by the operator. To solve for the amount of pressure provided by the ventilator (P_{V} or target airway pressure), simply divide the second equation by the first equation listed above to get the following equation:

P_{V}(t)=P_{P}(t)*(k/(1.0−k))

While an effort-based breath type is very beneficial to the patient, the computational cycle of the target pressure may be behind the actual demand of the patient. For example, the target pressure may be behind the actual demand by 5 to 50 milliseconds or more due at least to signal and/or calculation delays. Further, in aggressively breathing patients this time gap may be even larger.

Additionally, in one example, inspiratory muscle pressure, Pₘᵤₛ, (i.e., one example of how patient effort may be calculated) is a time-variant excitation function with inter- and intra-subject variations. It is hypothesized that in normal subjects Pₘᵤₛ is dependent on breath rate, inspiration time, and characteristic metrics of inspiratory pressure waveform. However, in actual patients, other factors related to demanded and expendable muscle energy may critically influence muscle pressure generation, which are not accounted for in effort-based breath types. For example, for a given peak inspiratory pressure, the maximum sustainable muscle pressure may be affected by factors impairing muscle blood flow (blood pressure, vasomotor tone, muscle tension in the off-phase), blood substrate concentration (glucose, free fatty acids), and the ability to extract source of energy from the blood. Thus, respiratory motor output may vary significantly in response to variations in metabolic rate, chemical stimuli, temperature, mechanical load, sleep state, and behavioral inputs. Moreover, the breath-by-breath variability in respiratory output could lead to tidal volumes varying by a factor of four or more. Accordingly, the patient may desire more or less pressure and/or breath than being delivered by the ventilator in an effort-based breath type.

Further, the delivered target pressure in the effort-based breath type has no set trajectory and is determined arbitrarily based on patient effort. For example, the trajectory can be determined every command cycle using estimated patient respiratory parameters, instantaneous inspiratory lung flow, patient-generated muscle pressure, and a clinician-set support setting. Therefore, inspiration and/or expiration may abruptly end or start based on these settings or parametric uncertainties and measurement and computational issues may cause morphological artifacts in the reference waveform trajectory. Patients, whether breathing aggressively, shallowly, or softly, typically exhibit a smooth trajectory from inspiration to expiration making any abrupt changes in the trajectory uncomfortable for the patient.

Further, while earlier trigger detection reduces trigger delays, the corresponding inspiration flow at this earlier moment of triggering may be initially too weak causing the algebraic magnitude of estimated lung flow to still be negative resulting in no delivery of a patient desired breath and/or the premature ending of a patient desired breath. Therefore, a weak triggering patient may not receive a breath and/or the amount of breath desired.

Accordingly, the current disclosure describes a model-based transformed proportional assist (MT-PA) breath type for ventilating a patient. The MT-PA breath type delivers a target pressure to the patient calculated based on a predetermined pressure trajectory model representing a well-defined waveform morphology and a support setting for a triggering patient. The predetermined trajectory prevents the patient from experiencing any abrupt inspiration and exhalation changes.

Further, the trajectory model may be based on clinically observed breath trajectories. For example, the trajectory may be based on clinically observed trajectories for specific breathing rates and/or types, such as aggressive, weak, or shallow breathing patients. In some embodiments, the trajectory may be based on clinically observed trajectories for patients with common diseases, such as chronic obstructive pulmonary disease (COPD), emphysema, or acute respiratory distress syndrome (ARDS). By utilizing a predetermined trajectory based on the patient and assigning or adaptively deriving appropriate quantitative values for the parameters of the model, the predetermined trajectory should already anticipate patient breath desires or be predictive of patient inspiratory muscle pressure. Accordingly, the predetermined trajectory should minimize or eliminate any gaps between actual patient demand and the calculated target pressure delivered and converge towards meeting patient demands.

In some embodiments, a multiplier may be applied to the at least one monitored parameter when the monitored parameter is below a predetermined threshold for triggering the delivery of a breath, such as a low or negative lung flow with positive (increasing) slope. The multiplier allows weak patients to receive desired breaths with pressure support. The predetermined trajectory in combination with the multiplier allows the ventilator to anticipate and deliver the type of breath desired by weak triggering patients.

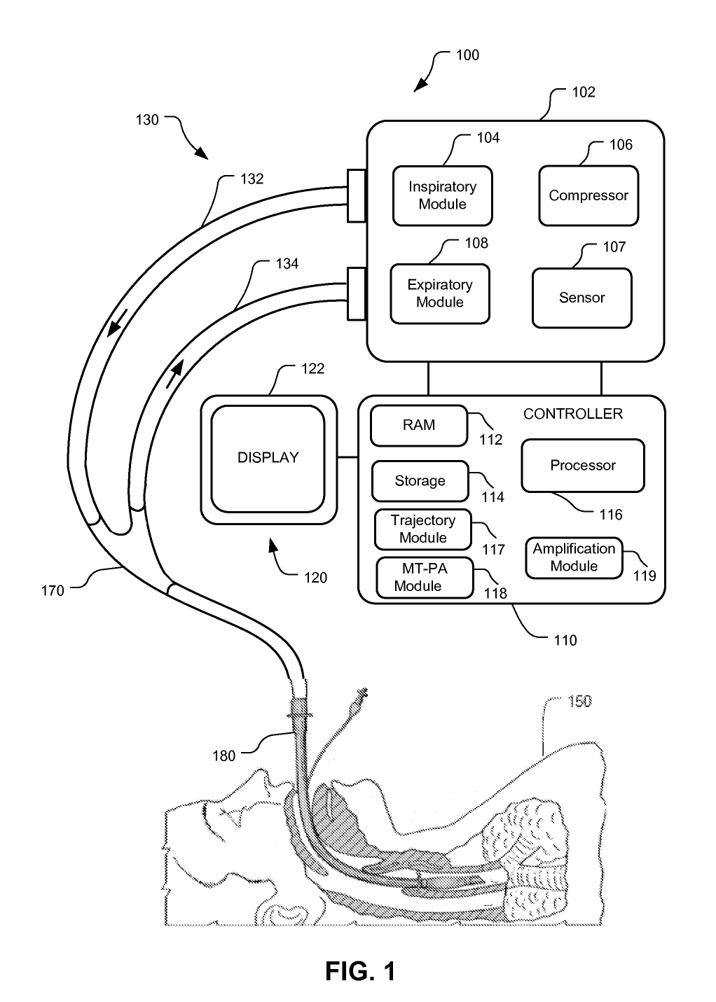

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endrotracheal tube, as shown) or a non-invasive (e.g., nasal mask) patient interface 180.

Ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endrotracheal tube) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 and the expiratory module 108 to provide a gas source for ventilatory support via inspiratory limb 132.

The inspiratory module 104 is configured to deliver gases to the patient 150 according to prescribed ventilatory settings. In some embodiments, inspiratory module 104 is configured to provide ventilation according to various breath types, e.g., via volume-control, pressure-control, MT-PA, or via any other suitable breath types.

The expiratory module 108 is configured to release gases from the patient's lungs according to prescribed ventilatory settings. Specifically, expiratory module 108 is associated with and/or controls an expiratory valve for releasing gases from the patient 150.

The ventilator 100 may also include one or more sensors 107 communicatively coupled to ventilator 100. The sensors 107 may be located in the pneumatic system 102, ventilation tubing system 130, and/or on the patient 150. The embodiment of FIG. 1 illustrates a sensor 107 in pneumatic system 102.

Sensors 107 may communicate with various components of ventilator 100, e.g., pneumatic system 102, other sensors 107, processor 116, trajectory module 117, MT-PA module 118, and any other suitable components and/or modules. In one embodiment, sensors 107 generate output and send this output to pneumatic system 102, other sensors 107, processor 116, trajectory module 117, MT-PA module 118, and any other suitable components and/or modules. Sensors 107 may employ any suitable sensory or derivative technique for monitoring one or more patient parameters or ventilator parameters associated with the ventilation of a patient 150. Sensors 107 may detect changes in patient parameters indicative of patient triggering, for example. Sensors 107 may be placed in any suitable location, e.g., within the ventilatory circuitry or other devices communicatively coupled to the ventilator 100. Further, sensors 107 may be placed in any suitable internal location, such as, within the ventilatory circuitry or within components or modules of ventilator 100. For example, sensors 107 may be coupled to the inspiratory and/or expiratory modules for detecting changes in, for example, circuit pressure and/or flow. In other examples, sensors 107 may be affixed to the ventilatory tubing or may be embedded in the tubing itself. According to some embodiments, sensors 107 may be provided at or near the lungs (or diaphragm) for detecting a pressure in the lungs. Additionally or alternatively, sensors 107 may be affixed or embedded in or near wye-fitting 170 and/or patient interface 180. Indeed, any sensory device useful for monitoring changes in measurable parameters during ventilatory treatment may be employed in accordance with embodiments described herein.

As should be appreciated, with reference to the Equation of Motion, ventilatory parameters are highly interrelated and, according to embodiments, may be either directly or indirectly monitored. That is, parameters may be directly monitored by one or more sensors 107, as described above, or may be indirectly monitored or estimated by derivation according to the Equation of Motion.

The pneumatic system 102 may include a variety of other components, including mixing modules, valves, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.).

In one embodiment, the operator interface 120 of the ventilator 100 includes a display 122 communicatively coupled to ventilator 100. Display 122 provides various input screens, for receiving clinician input, and various display screens, for presenting useful information to the clinician. In one embodiment, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 100 may be provided, for instance by a wheel, keyboard, mouse, or other suitable interactive device. Thus, operator interface 120 may accept commands and input through display 122. Display 122 may also provide useful information in the form of various ventilatory data regarding the physical condition of a patient 150. The useful information may be derived by the ventilator 100, based on data collected by a processor 116, and the useful information may be displayed to the clinician in the form of graphs, wave representations, pie graphs, text, or other suitable forms of graphic display. For example, patient data may be displayed on the GUI and/or display 122. Additionally or alternatively, patient data may be communicated to a remote monitoring system coupled via any suitable means to the ventilator 100.

Controller 110 may include memory 112, one or more processors 116, storage 114, and/or other components of the type commonly found in command and control computing devices. Controller 110 may further include a trajectory module 117, a MT-PA module 118, and amplification module 119 configured to deliver gases to the patient 150 according to prescribed breath types as illustrated in FIG. 1. In alternative embodiments, the trajectory module 117, the MT-PA module 118, and the amplification module 119 may be located in other components of the ventilator 100, such as the pressure generating system 102 (also known as the pneumatic system 102).

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

The inspiratory module 104 receives a breath type from the MT-PA module 118 and a predetermined trajectory for the breath type from the trajectory module 117. In some embodiments, the MT-PA module 118 and/or the trajectory module 117 are part of the controller 110 as illustrated in FIG. 1. In other embodiments, the MT-PA module 118 and/or the trajectory module 117 are part of the processor 116, pneumatic system 102, and/or a separate computing device in communication with the ventilator 100.

The trajectory module 117 receives a predetermined trajectory for the MT-PA breath type. Further, in some embodiments, the trajectory module 117 or any other suitable component of the ventilator 100, such as the processor 116, controller 110, or pneumatic system 102, estimates patient parameters based on the at least one monitored patient parameter from the sensor(s) 107. In some embodiments, the estimated parameters are calculated by entering the monitored parameters into the Equation of Motion. In further embodiments, the monitored patient parameters are inspiratory flow and/or net flow. In some embodiments, the estimated patient parameters are at least one of resistance, elastance, and/or compliance.

The predetermined trajectory is a model trajectory. In some embodiments, the predetermined trajectory is based on clinically observed trajectories for patients with specific breathing rates/types or for patients suffering from a specific disease state. For example, the predetermined trajectory may be based on clinically observed trajectories for patients with shallow breathing, aggressive breathing, COPD, ARDS, etc. The predetermined trajectory based on clinically observed data anticipates the trajectory desired by the patient 150 by utilizing the patient's particular characteristics, such as disease state or rate of breathing. In some embodiments the trajectory is a sinusoidal, a modified sinusoidal, and/or a modified square model wave-form. Accordingly, in some embodiments, the predetermined trajectory is predictive of patient inspiratory muscle pressure.

In one embodiment, the predetermined trajectory is a sinusoidal model as illustrated below, which approximates actual clinically-observed inspiratory muscle pressures:

$${P}_{musi} = - {P}_{max} ( 1 - \frac{t}{{t}_{v}} ) sin ( \frac{π t}{{t}_{v}} ) .$$

Pₘᵤₛᵢ represents the magnitude of the negative muscle pressure generated by inspiratory muscles and is therefore equivalent to the amount of pressure that must be provided by the patient (or P_{P}) at a time t. Accordingly, Pₘᵤₛᵢ can be substituted into the PA support setting equations illustrated above as P_{P}(t) to solve for a target airway pressure at the time t (or P_{V}(t)) as illustrated below:

$${P}_{V} ( t ) = {P}_{max} ( 1 - \frac{t}{{t}_{v}} ) sin ( \frac{π t}{{t}_{v}} ) * ( k / ( 1.0 - k ) ) .$$

Pₘₐₓ represents the maximum amount of muscle pressure (representing patient effort) that the patient should exert, tᵥ represents ventilator detected inspiration duration, and t represents elapsed breath time varying between 0 and the total sum of inspiration and expiration periods in the above equation. The Pₘₐₓ and tᵥ may be input by the operator, selectable by the operator from a group of predetermined values, and/or selected or estimated by the ventilator based on monitored patient parameters, estimated patient parameters, and ventilator parameters. In some embodiments, the parameters of the above equation are adjusted based on monitored patient parameters indicating and trending breathing behavior in conjunction with optimization algorithms to achieve a quantitatively defined objective (e.g., minimize work of breathing, minimize fatigue, optimize oxygenation, etc.). The sinusoidal model predetermined pressure trajectory illustrated above is merely exemplary and is not meant to be limiting. Any suitable predetermined pressure trajectory for a patient on a ventilator may be utilized by the trajectory module 117.

The predetermined trajectory may be input by an operator, selected by an operator from a group of predetermined trajectories, determined by the ventilator 100, or selected by the ventilator 100 from a group of predetermined trajectories. The ventilator 100 may select or determine the predetermined trajectory by monitoring patient parameters, estimating patient parameters, and by monitoring ventilator parameters. In some embodiments, ventilator parameters include settings such as tidal volume, fractional inspired oxygen, and/or positive end-expiratory pressure. In some embodiments, the ventilator 100 determines the predetermined trajectory or adjusts the values assigned to the parameters of the predetermined trajectory on a breath-by-breath basis. In some embodiments, the ventilator 100 utilizes this data to determine if the patient 150 suffers from a specific disease or to determine the patient breath rate, such as if the patient 150 is breathing aggressively or shallowly. During these embodiments, the ventilator 100 determines the best trajectory to utilize based on, for example, the breath rate, tidal volumes, and disease state determinations.

In other embodiments, once a predetermined trajectory is being delivered to the patient 150, the ventilator 100 determines that the delivered trajectory is improper and should be changed. If the ventilator 100 detects that predetermined trajectory should be adjusted or changed, the ventilator 100 may notify the operator. The operator may be notified by the ventilator 100 with any visual, audio, and/or vibrational notification systems and/or methods. For example, an alarm may sound and a prompt explaining the need for a change in the predetermined trajectory may be displayed. In some embodiments, if the ventilator 100 detects that the predetermined trajectory should be adjusted or changed, the ventilator 100 determines the proper trajectory that should be utilized. In some embodiments, the ventilator 100 determines that the predetermined trajectory needs to be changed and/or what the proper trajectory should be by monitoring patient parameters, estimating patient parameters, and/or monitoring ventilator parameters. In further embodiments, the ventilator 100 determines if the predetermined trajectory needs to be changed and modified on a breath-by-breath basis or over a definite window of time or patient state (e.g., sleep or wakefulness).

For example, the ventilator may estimate patient effort by monitoring flow and derive a target pressure based on the estimated patient effort according to a PA breath type and then compare the estimated patient effort and/or derived target pressure to the predetermined trajectory. If the estimated patient effort and/or derived target pressure vary too much from the predetermined trajectory, the ventilator 100 may modify specific parameters of the predetermined trajectory, such as Pₘₐₓ and tᵥ, or may change the predetermined trajectory to an entirely different model based on these comparison results.

In these embodiments, the ventilator 100 may notify the operator of the needed change and recommend a new predetermined determined trajectory. In these embodiments, the ventilator 100 may notify the operator of the needed change and recommend new parameters for the predetermined determined trajectory. Alternatively, in these embodiments, the ventilator 100 may simply change the predetermined trajectory to a new predetermined trajectory. Further, in these embodiments, the ventilator 100 may simply change the parameters of the predetermined trajectory. In one embodiment, the trajectory parameters include maximum or peak amplitude, phase angle, Pₘₐₓ, and/or tᵥ.

Initiation and execution of an MT-PA breath type has two operation prerequisites: (1) detection of an inspiratory trigger; and (2) determining and commanding a reference airway pressure trajectory to the controller for the duration of the just-started inspiration. A patient trigger is calculated based on a measured or monitored patient inspiration flow. In addition, the sensitivity of the ventilator 100 to changes in a monitored patient parameter, such as flow, may be adjusted such that the ventilator 100 may properly detect changes in the monitored parameter. For example, the lower the pressure or flow change threshold setting, the more sensitive the ventilator 100 may be to a patient initiated trigger. However, each ventilator 100 will have a minimum measurable inspiration flow and thereby have a change in flow that the ventilator 100 can not detect. Accordingly, a monitored parameter below a minimum measurable value will not be detected by the ventilator 100.

Any suitable type of triggering detection for determining a patient trigger may be utilized by the ventilation system, such as nasal detection, diaphragm detection, and/or brain signal detection. Further, the ventilator 100 may detect patient triggering via a pressure-monitoring method, a flow-monitoring method, direct or indirect measurement of neuromuscular signals, or any other suitable method. Sensors 107 suitable for this detection may include any suitable sensing device as known by a person of skill in the art for a ventilator.

According to an embodiment, a pressure-triggering method may involve the ventilator 100 monitoring the circuit pressure, and detecting a slight drop in circuit pressure. The slight drop in circuit pressure may indicate that the patient's respiratory muscles are creating a slight negative pressure that in turn generates a pressure gradient between the patient's lungs and the airway opening in an effort to inspire. The ventilator 100 may interpret the slight drop in circuit pressure as a patient trigger and may consequently initiate inspiration by delivering respiratory gases.

Alternatively, the ventilator 100 may detect a flow-triggered event. Specifically, the ventilator 100 may monitor the circuit flow, as described above. If the ventilator 100 detects a slight drop in the base flow through the exhalation module during exhalation, this may indicate, again, that the patient 150 is attempting to inspire. In this case, the ventilator 100 is detecting a drop in bias flow (or baseline flow) attributable to a slight redirection of gases into the patient's lungs (in response to a slightly negative pressure gradient as discussed above). Bias flow refers to a constant flow existing in the circuit during exhalation that enables the ventilator 100 to detect expiratory flow changes and patient triggering.

The MT-PA module 118 sends a MT-PA breath type to the inspiratory module 104. The MT-PA breath type refers to a type of ventilation in which the ventilator 100 acts as an inspiratory amplifier that provides pressure support to the patient. The degree of amplification (the “support setting”) is set by an operator, for example as a percentage based on the patient's effort. For example, if the operator sets the support setting to 30%, then the ventilator provides 30% of the desired pressure to the patient and the patient must provide the remaining 70% of the desired pressure.

The MT-PA breath type determines a target pressure by utilizing the support setting in combination with predetermined trajectory. The predetermined trajectory replaces the Pₚ(t) parameter in the equation listed below for determining a target pressure (or P_{V}(t)) at a time t:

P_{V}(t)=P_{P}(t)*(k/(1.0−k)).

In one implementation, every computational cycle (e.g., 5 milliseconds, 10 milliseconds, etc.), the ventilator calculates a target pressure, based on the support setting and the predetermined trajectory. In one embodiment, as discussed above, the predetermined trajectory is the following:

$${P}_{musi} = - {P}_{max} ( 1 - \frac{t}{{t}_{v}} ) sin ( \frac{π t}{{t}_{v}} ) .$$

Accordingly, the target pressure (P_{V}) at time t is solved for by utilizing the following

$${P}_{V} ( t ) = {P}_{max} ( 1 - \frac{t}{{t}_{v}} ) sin ( \frac{π t}{{t}_{v}} ) * ( k / ( 1.0 - k ) ) .$$

The MT-PA module 118 begins inspiratory assist when a trigger is detected and/or when the at least one monitored parameter is detected by the MT-PA module 118. However, if the patient ceases triggering inspiration, the assist also ceases. Accordingly, in some embodiments, the MT-PA module 118 includes a safety feature that has the ventilator 100 deliver a breath to the patient or switches the breath type to a non-spontaneous breath type if a patient trigger is not detected for a set period of time or based on the occurrence of a set event. This safety feature ensures that if a patient stops triggering, the patient will not stop receiving ventilation by the medical ventilator.

Further, this functionality is often a problem for weak patients with weak detected triggers. A weak detected trigger may not generate a measurable flow for the delivery of a patient breath. Once a measurable, positive inspiration flow is detected by the ventilator, the ventilator applies the calculated target pressure to deliver a proportion (determined by the support setting) of the total demand as determined by the predetermined pressure trajectory.

Accordingly, in some embodiments, the ventilator 100 utilizes an amplification module 119. The amplification module 119 determines that a detected patient trigger is generated in the presence of a negative inspiration flow as measured by the ventilator (indicating that the patient is still exhaling) or an inspiration flow below a predetermined threshold that typically would not provide a desired breath to the patient. The amplification module 119 applies a multiplier or multiplicative transformation to the monitored flow below the predetermined threshold to form an adjusted or amplified flow. As used herein, the terms “multiplier” and “multiplicative transformation” are considered to be interchangeable in the present disclosure and in the claims. While “multiplier” and “multiplicative transformation” refer to different values, it is understood by a person of skill in the art that each may be used for the purposes of this disclosure and for the purposes of the claims. The adjusted or amplified flow is utilized by the MT-PA module 118 to determine a patient trigger for delivering a pressure supported breath to the patient. Accordingly, the amplification module 119 allows weak patients to trigger a pressure supported breath. Since, the predetermined trajectory provided by the trajectory module 117 anticipates the desired trajectory of the patient, the pressure supported breath provided by the amplification module 119 should be close or equivalent to the breath desired by the weakly triggering patient.

In some embodiments, the amplification module 119 is activated by the operator. In other embodiments, the amplification module 119 is activated by the ventilator. In some embodiments, the amplification module 119 is deactivated by the ventilator 100 and in other embodiments, the amplification module 119 is deactivated be the operator. In some embodiments, the amplification module 119 is activated and/or deactivated by the ventilator 100 based on the monitoring of ventilator parameters, monitoring of patient parameters, the estimating of patient parameters and/or the occurrence of a predetermined event. For example, the predetermined event may include detecting a predetermined number of monitored parameters above or below the predetermined threshold in a predetermined amount of time. In another example, the predetermined event may be detecting a predetermined number of consecutive measured monitored parameters above or below the predetermined threshold. In a further example, the predetermined event is the detection of a predetermined number of monitored parameters above or below the predetermined threshold in a predetermined number of breaths.

The multiplier utilized by the amplification module 119 may be input by an operator, selected by an operator from a group of predetermined multipliers, determined by the ventilator 100, or selected by the ventilator 100 from a group of predetermined multipliers. The ventilator 100 may select or determine the multiplier by monitoring patient parameters, estimating patient parameters, and by monitoring ventilator parameters. In some embodiments, the multiplier is adaptively modified by the ventilator 100 on a breath-by-breath basis based on at least one of the monitored patient parameters, the estimated patient parameters, and/or from monitoring ventilator parameters.

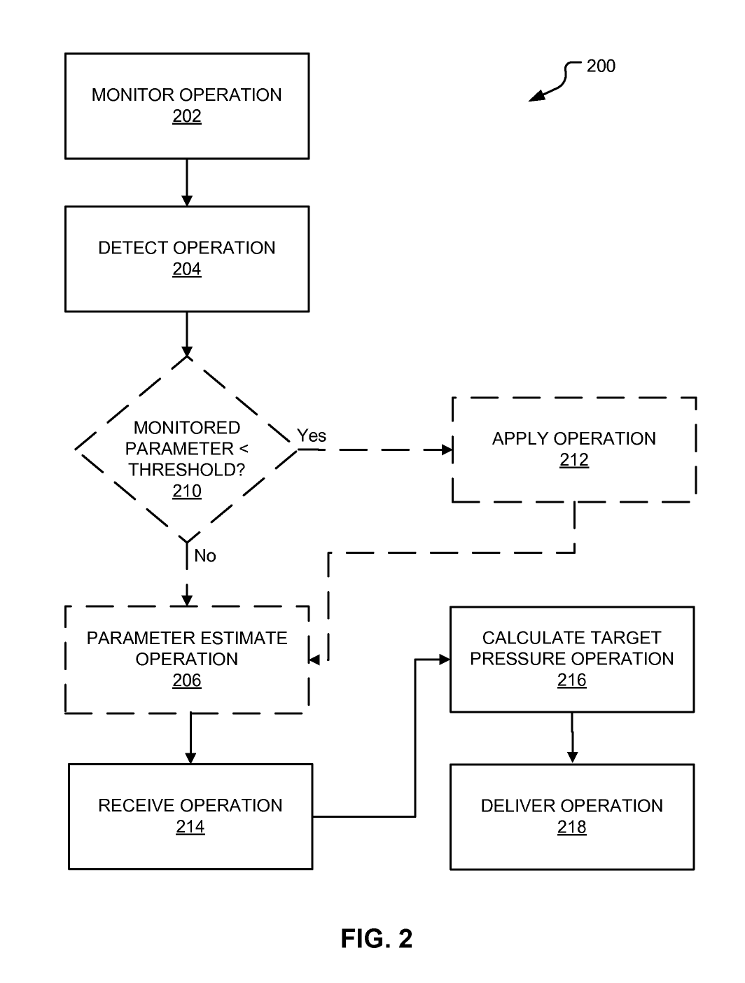

FIG. 2 illustrates an embodiment of a method 200 for ventilating a patient with a ventilator that utilizes a MT-PA breath type. The MT-PA breath type has a predetermined trajectory, so the target pressure is calculated based on the predetermined trajectory and a support setting. The predetermined trajectory prevents the patient from experiencing artifactual waveform patterns and/or any abrupt inspiration and exhalation changes. Further, because the predetermined trajectory is based on clinically observed trajectories, the predetermined trajectory may be adaptively adjusted on an ongoing basis to minimize any gap between actual patient demand and the calculated target pressure delivered.

As illustrated, method 200 includes a monitoring operation 202. During the monitoring operation 202, the ventilator monitors patient parameters. In some embodiments, the patient parameters include inspiratory lung flow, net lung flow, and/or airway pressure. The monitoring operation 202 may be performed by sensors and data acquisition subsystems. The sensors may include any suitable sensing device as known by a person of skill in the art for a ventilator. In some embodiments, the sensors are located in the pneumatic system, the breathing circuit, and/or on the patient. In some embodiments, the ventilator during the monitoring operation 202 monitors the inspiration flow every computational cycle (e.g., 2 milliseconds, 5 milliseconds, 10 milliseconds, etc.) during the delivery of the control pressure.

Further, method 200 includes a detection operation 204. During the detection operation 204, the ventilator detects an inspiratory trigger. The inspiratory trigger may be detected by any suitable method for detecting an inspiratory trigger, such as nasal detection, diaphragm detection, brain signal detection, pressure monitoring detection, and/or flow monitoring detection. The triggering detection is based on sensor readings. Sensors may include any suitable sensing device as known by a person of skill in the art for a ventilator.

In some embodiments, method 200 includes a parameter estimation operation 206. During the parameter estimation operation 206, the ventilator estimates patient parameters based on the measurements directly or indirectly related to monitored patient parameters. In some embodiments, the estimated patient parameters include lung compliance (inverse of elastance) and/or lung/airway resistance. In further embodiments, the estimated lung compliance, lung elastance and/or lung/airway resistance are estimated based on monitored flow and/or the Equation of Motion. The estimated patient parameters may be estimated by any suitable processor found in the ventilator. In some embodiments, the estimated patient parameters are calculated by a controller, a pneumatic system, and/or a separate computing device operatively connected to the ventilator.

As illustrated, method 200 includes a receiving operation 214. During the receiving operation 214, the ventilator receives a predetermined pressure trajectory. The predetermined trajectory is a model trajectory. In some embodiments the predetermined trajectory is based on clinically observed trajectories for patients with specific breathing rates/types or for patients suffering from a specific disease state. For example, the predetermined trajectory may be for patients with shallow breathing, aggressive breathing, COPD, ARDS, etc. The predetermined trajectory anticipates the trajectory generated by the patient (according to a PA breath type calculation) by utilizing the patient's particular characteristics, such as disease state or rate of breathing. In some embodiments the trajectory may be a sinusoidal, a modified sinusoidal, and/or a modified square model wave-form.

In one embodiment, the predetermined trajectory received by the ventilator during the receiving operation 214 is a model sinusoidal wave-form as defined below:

Pₘᵤₛᵢ=−Pₘₐₓ(1−(t/tᵥ))sin(πt/tᵥ).

Pₘᵤₛᵢ represents the magnitude of the negative muscle pressure generated by inspiratory muscles and is therefore equivalent to the amount of pressure that must be provided by the patient (or P_{P}) at a time t. Accordingly, Pₘᵤₛᵢ can be substituted into the PA support setting equations illustrated above as Pₚ(t) to solve for a target pressure (or P_{V}(t)) as illustrated below:

$${P}_{V} ( t ) = {P}_{max} ( 1 - \frac{t}{{t}_{v}} ) sin ( \frac{π t}{{t}_{v}} ) * ( k / ( 1.0 - k ) ) .$$

As discussed above, the Pₘₐₓ and tᵥ may input by the operator, selected by the operator from a group of predetermined values, and/or selected or estimated by the ventilator based on monitored patient parameters, estimated patient parameters, and ventilator parameters. The sinusoidal model predetermined pressure trajectory illustrated above is merely exemplary and is not meant to be limiting. Any suitable predetermined pressure trajectory for a patient on a ventilator in an effort-based breath type may be received by the ventilator during receiving operation 214.

The predetermined trajectory may be received from input by an operator, a selection by an operator from a group of predetermined trajectories, the ventilator, or a selection by the ventilator from a group of predetermined trajectories. The ventilator may select or determine the predetermined trajectory from the monitored patient parameters, the estimated patient parameters, and from monitoring ventilator parameters. In some embodiments, the ventilator may select or determine the predetermined trajectory from the monitored patient parameters, the estimated patient parameters, and from monitoring ventilator parameters on a breath-by-breath basis. In some embodiments, the ventilator utilizes this data to determine if the patient suffers from a specific disease or if the patient is exhibiting symptoms consistent with a condition of interest to be reported to the clinician by the ventilator. During these embodiments, the ventilator determines the best trajectory to utilize based on the breath rate and/or disease state determinations.

Next, method 200 includes a calculating operation 216. During the calculating operation 216, the ventilator calculates a target airway pressure based on the predetermined pressure trajectory and a support setting. The target pressure is calculated for a point in the ventilation circuit that is proximal to the lung and would best assist the patient's inspiratory muscles to the degree selected by the operator as the support setting. In one embodiment, the support setting (or the degree of amplification) is set by an operator, for example as a percentage based on the patient's effort. The predetermined trajectory replaces the amount of pressure that must be provided by the patient at a time t (or Pₚ(t) in the equation listed below for determining a target airway pressure (or Pᵥ(t)) at a tune t:

P_{V}(t)=P_{P}(t)*(k/(1.0−k)).

Method 200 also includes a delivery operation 218. During the delivery operation 218, the ventilator delivers the target airway pressure to a patient based on the detected inspiratory trigger. A patient trigger is calculated based on the at least one monitored parameter, such as inspiration flow. In some embodiments, sensors, such as flow sensors, may detect changes in patient parameters indicative of patient triggering. In addition, the sensitivity of the ventilator to changes in the at least one monitored parameter may be adjusted such that the ventilator may properly detect changes in flow. For example, the lower the pressure or flow change threshold setting, the more sensitive the ventilator may be to a patient initiated trigger. However, each ventilator will have a minimum measurable monitored parameter value and thereby a change in the monitored parameter that the ventilator can not detect. A monitored parameter below this minimum change will not be detected by the ventilator.

Advanced, sophisticated triggering technologies detect initiation of inspiratory efforts more efficiently. However, while earlier detection of an inspiratory effort reduces trigger delays, the inspiratory flow at the time of triggering may initially be so weak that the ventilator may not be able to measure its magnitude. Further, the inspiratory flow at the time of triggering may be negative if the patient triggers inhalation while still exhaling. Further, if the patient ceases inspiration, the inspiration flow is zero. Therefore, weak patients with weak triggers may not receive a desired breath.

Accordingly, in some embodiments, method 200 further includes a decision operation 210 and an application operation 212 after the performance of the detection operation 204 by the ventilator. The decision operation 210 determines if the at least one monitored parameter, such as flow, is less than a predetermined threshold. If the ventilator during the decision operation 210 determines that the at least one monitored parameter is equal to or greater than a predetermined threshold, then the ventilator selects to perform receiving operation 214. If the ventilator during the decision operation 210 determines that the monitored parameter is less than the predetermined threshold, then the ventilator selects to perform an application operation 212. In some embodiments, the predetermined threshold is a monitored parameter, such as flow, that is too low for the delivery of a breath by the ventilator during the delivery operation 218.

In further embodiments, method 200 includes the application operation 212. The ventilator during application operation 212 applies a multiplier or a multiplicative transformation to the monitored patient parameter. Accordingly, the ventilator during the delivery operation 218 utilizes the monitored parameter, such as flow, after the monitored parameter has been adjusted or amplified by the ventilator during the application operation 212. The multiplier may be input by an operator, selected by an operator from a group of predetermined multipliers, from the ventilator, or selected by the ventilator from a group of predetermined multipliers. The ventilator may select or determine the multiplier from the monitored patient parameters, the estimated patient parameters, and/or from monitoring ventilator parameters. In some embodiments, the multiplier is adaptively modified by the ventilator on a breath-by-breath basis based on at least one of the monitored patient parameters, the estimated patient parameters, and/or from monitoring ventilator parameters.

In some embodiments, the decision operation 210 and application operation 212 are utilized by the ventilator during method 200 when activated by the operator. In other embodiments, the decision operation 210 and application operation 212 of method 200 are activated by the ventilator. In some embodiments, the decision operation 210 and application operation 212 of method 200 are deactivated by the ventilator and in other embodiments, the decision operation 210 and application operation 212 of method 200 are deactivated by the operator. In some embodiments, the decision operation 210 and application operation 212 of method 200 are activated and/or deactivated by the ventilator based on the monitoring of ventilator parameters, monitoring of patient parameters, the estimating of patient parameters and/or the occurrence of a predetermined event. For example, the predetermined event may include detecting a predetermined number of patient triggers with a monitored parameter above or below the predetermined threshold in a predetermined amount of time. In another example, the predetermined event may be detecting a predetermined number of consecutive patient triggers above or below the predetermined threshold. In a further example, the predetermined determined event is the detection of a predetermined number of triggers with a flow above or below the predetermined threshold in a predetermined number of breaths.

In other embodiments, method 200 includes a display operation. The ventilator during the display operation displays any suitable information for display on a ventilator. In one embodiment, the display operation displays at least one of the predetermined trajectory, a recommended trajectory, a pressure waveform, the target pressure, a support setting, the monitored patient parameters, a multiplier, and/or estimated patient parameters.

In other embodiments, method 200 includes a trajectory decision operation after the ventilator performs the delivery operation 218. The ventilator during the trajectory decision operation determines if the predetermined trajectory is improper and should be changed and/or adjusted. In some embodiments, the ventilator determines that the predetermined trajectory needs to be changed based on the monitored patient parameters, the estimated patient parameters, and/or monitored ventilatory parameters. In some embodiments, the monitored patient parameters, the estimated patient parameters, and/or monitored ventilatory parameters are utilized in accordance with an optimization algorithm to determine that the predetermined trajectory needs to be changed. In some embodiments, the ventilator determines that the predetermined trajectory needs to be changed or adjusted on a breath-by-breath basis. If the ventilator during the trajectory decision operation detects that predetermined trajectory does not need to be changed, the ventilator selects to perform monitoring operation 202. If the ventilator during the trajectory decision operation detects that the predetermined trajectory should be changed, the ventilator selects to perform at least one of a notification operation, a recommendation operation, and/or change operation.

In some embodiments, method 200 includes a notification operation. The ventilator during the notification operation notifies the operator with any suitable visual, audio, or vibrational notification methods for a ventilator system that the predetermined trajectory is improper. For example, the ventilator during the notification operation may sound an alarm and display a prompt explaining the need to change the predetermined trajectory.

In some embodiments, method 200 includes a recommendation operation. In some embodiments, the ventilator during the recommendation operation recommends that the operator change the predetermined trajectory to a different predetermined trajectory. In other embodiments, the ventilator during the recommendation operation recommends that the operator change inputs of the predetermined trajectory to different values, such as Pₘₐₓ, tᵥ, peak amplitude, inspiration duration, and/or phase angle. The ventilator during the recommendation operation determines a better predetermined trajectory or inputs of the predetermined trajectory by monitoring patient parameters, estimating patient parameters, and/or monitoring ventilator parameters. In some embodiments, the ventilator during the recommendation operation determines a better predetermined trajectory or inputs of the predetermined trajectory on a breath-by-breath basis. The ventilator recommends a better predetermined trajectory or input by any suitable notification method for a ventilator, such as any visual, audio, or vibrational notification method.

In some embodiments, method 200 includes a change operation. In some embodiments, the ventilator during the change operation changes the predetermined trajectory to a different predetermined trajectory. In other embodiments, the ventilator during the change operation changes the inputs of the predetermined trajectory. The ventilator during the change operation determines a more appropriate predetermined trajectory or inputs for the predetermined trajectory by utilizing the monitored patient parameters, the estimated patient parameters, and/or monitored ventilator parameters. In some embodiments, the ventilator during the change operation determines a more appropriate predetermined trajectory or inputs for the predetermined trajectory on a breath-by-breath basis.

In some embodiments, a microprocessor-based ventilator that accesses a computer-readable medium having computer-executable instructions for performing the method of ventilating a patient with a medical ventilator is disclosed. This method includes repeatedly performing the steps disclosed in method 200 above and/or as illustrated in FIG. 2.

In some embodiments, the ventilator system includes: means for monitoring at least one patient parameter; means for detecting an inspiratory trigger based on the at least one monitored patient parameter; means for receiving a predetermined pressure trajectory; means for calculating a target pressure based at least on the predetermined pressure trajectory and a support setting; and means for delivering the target pressure to a patient based on the detected inspiratory trigger.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for ventilating a patient with a ventilator comprising:
monitoring at least one patient parameter;
detecting an inspiratory trigger based on the at least one monitored patient parameter;
receiving a predetermined pressure trajectory;
calculating a target pressure based at least on the predetermined pressure trajectory and a support setting; and
delivering the target pressure to a patient based on the detected inspiratory trigger.

2. The method of claim 1, wherein the predetermined pressure trajectory includes at least one of an inspiratory duration, a phase angle, and a peak amplitude.

3. The method of claim 2, wherein at least one of the inspiratory duration, the phase angle, and set peak amplitude are at least one of selected by an operator, input by an operator, and/or determined by the ventilator.

4. The method of claim 1, further comprising:
estimating patient parameters based on the at least one monitored patient parameter; and
determining that the predetermined pressure trajectory is improper for the patient based on at least one of the estimated patient parameters, the at least one monitored patient parameter, and ventilator parameters.

5. The method of claim 4, further comprising:
adjusting at least one of a Pₘₐₓ and a tᵥ of the predetermined pressure trajectory based on the step of determining that the predetermined pressure trajectory is improper.

6. The method of claim 4, further comprising:
changing the predetermined pressure trajectory to a different model based on the step of determining that the predetermined pressure trajectory is improper.

7. The method of claim 4, further comprising:
displaying a recommended change in at least one of an inspiratory duration, a phase angle, a peak amplitude, a Pₘₐₓ, a tᵥ and the predetermined pressure trajectory based on the step of determining that the predetermined pressure trajectory is improper.

8. The method of claim 4, wherein the estimated patient parameters are compliance and resistance.

9. The method of claim 1, wherein the predetermined pressure trajectory is received from at least one of operator selection from a group of predetermined trajectories, input by an operator, the ventilator based on at least one of the at least one monitored patient parameter, estimated patient parameters, and ventilator parameters, and ventilator selection from a group of predetermined trajectories based on at least one of the at least one monitored patient parameter, the estimated patient parameters, and the ventilator parameters.

10. The method of claim 1, further comprising:
determining that the at least one monitored patient parameter of the inspiratory trigger is below a predetermined threshold, wherein the at least one monitored patient parameter is a monitored flow; and
applying a multiplier to the monitored flow,
wherein the step of detecting the inspiratory trigger is based on the monitored flow after the monitored flow has been amplified by the multiplier.

11. The method of claim 10, wherein the multiplier is at least one of selectable by an operator from a group of predetermined multipliers, input by the operator, determined by the ventilator based on at least one of the at least one monitored patient parameter, the estimated patient parameters, and ventilator parameters, and selected by the ventilator from a group of predetermined multipliers based on at least one of the at least one monitored patient parameter, the estimated patient parameters, and the ventilator parameters.

12. The method of claim 10, wherein the multiplier is adaptively modified by the ventilator on a breath-by-breath basis based on at least one of the at least one monitored patient parameter, the estimated patient parameters, and ventilator parameters.

13. The method of claim 1, wherein the at least one monitored patient parameter is inspiratory flow.

14. The method of claim 1, further comprising:
displaying at least one of the predetermined pressure trajectory, a recommended pressure trajectory, a pressure waveform, the target pressure, the at least one monitored patient parameter, the support setting, a multiplier, and estimated patient parameters.

15. The method of claim 1, wherein the support setting is input by an operator.

16. A ventilator system comprising:
a pressure generating system adapted to generate a flow of breathing gas;
a ventilation tubing system including a patient interface for connecting the pressure generating system to a patient;
one or more sensors operatively coupled to at least one of the pressure generating system, the patient, and the ventilation tubing system, wherein at least one sensor is capable of generating an output indicative of an inspiration flow;
a trajectory module determines a pressure trajectory;
a MT-PA module, the MT-PA module calculates at least one target pressure based at least on the pressure trajectory and a support setting and utilizes the output indicative of the inspiration flow to determine a patient trigger for delivery of a breath to the patient; and
a processor in communication with the pressure generating system, the one or more sensors, the trajectory module, and the MT-PA module.

17. The ventilator system of claim 16, further comprising:
an amplification module, the amplification module determines that the output indicative of the inspiration flow is below a predetermined threshold and applies a multiplier to the output to form an amplified output, wherein the output utilized by MT-PA module is the amplified output.

18. The ventilator system of claim 16, further comprising:
a display in communication with at least one of the pressure generating system, the one or more sensors, the MT-PA module, the trajectory module, the processor, the MT-PA module, and an amplification module.

19. The ventilator system of claim 16, wherein the trajectory module determines if the pressure trajectory is proper based on parameters monitored by the one or more sensors, estimated parameters derived from the parameters monitored by the one or more sensors, and ventilator parameters.

20. The ventilator system of claim 19, wherein the trajectory module adjusts the pressure trajectory based on the parameters monitored by the one or more sensors, the parameters estimated from the parameters monitored by the one or more sensors, and the ventilator parameters.

21. The ventilator system of claim 19, wherein the trajectory module changes the pressure trajectory based on the parameters monitored by the one or more sensors, the parameters estimated from the parameters monitored by the one or more sensors, and the ventilator parameters.

22. A computer-readable medium having compute-executable instructions for performing a method of ventilating a patient with a ventilator, the method comprising:
repeatedly monitoring at least one patient parameter;
repeatedly detecting an inspiratory trigger based on the at least one monitored patient parameter;
repeatedly receiving a predetermined pressure trajectory;
repeatedly calculating a target pressure based at least on the predetermined pressure trajectory and a support setting; and
repeatedly delivering the target pressure to a patient based on the detected inspiratory trigger.

23. A ventilator system, comprising:
means for monitoring at least one patient parameter;
means for detecting an inspiratory trigger based on the at least one monitored patient parameter;
means for receiving a predetermined pressure trajectory;
means for calculating a target pressure based at least on the predetermined pressure trajectory and a support setting; and
means for delivering the target pressure to a patient based on the detected inspiratory trigger.
